(19) United States
(12) Patent Application Publication
(10) Pub. No.: US 2011/0152896 A1
(43) Pub. Date: Jun. 23, 2011

(54) Patch Plug for Extended Coverage During and After Hernia Surgery and Method of Anchoring Patch Plug

(76) Inventor: Jerry G. Purvis, JR., Circleville, OH (US)

(21) Appl. No.: 12/974,671

(22) Filed: Dec. 21, 2010

Related U.S. Application Data
(60) Provisional application No. 61/288,432, filed on Dec. 21, 2009.

(51) Int. Cl. A61B 17/00

(52) U.S. Cl. 606/151

(57) ABSTRACT

A system and method for repairing hernias in humans. The system includes a surgical mesh onlay patch to which a conical mesh plug is mounted. The mesh plug is elastic to permit the pre-compressed plug to be inserted into the deep inguinal ring and then expand upon release, which seats the plug's outer surfaces against the sidewall of the deep inguinal ring. The expanded plug holds the system in place while legs formed on the onlay patch are brought around the cord and attached together, and other attachments are made to the soft tissue of the inguinal canal floor. Preferably a growth-encouraging substance is applied to the exterior of the system to enhance attachment to surrounding tissue.

### BACKGROUND OF THE INVENTION

A hernia occurs when an organ or tissue, commonly a loop of the intestines A (see FIGS. 4B-4D), squeezes through a hole or a weak spot in a surrounding muscle or connective tissue called fascia. Hernias are caused by a combination of pressure and an opening or weakness of muscle or fascia. The pressure pushes an organ or tissue through the opening or weak spot in the muscle or fascia.

Most hernias are inguinal, and most occur in men because of a natural weakness in this area. In an inguinal hernia, an intra-abdominal organ, such as omentum, intestine, bladder or ovary can protrude into the inguinal canal in the groin. The inguinal canal is a 4 cm long tube-like space within the lower lateral part of the abdominal wall. It is here that the testes, which develop in the abdominal cavity, descend down into the scrotum. The extension of the testes through this region stretches and pulls this area of the abdominal wall, thereby making it weaker. This weakness is especially pronounced at the areas of the entrance and exit to the canal, which are the deep (also called the “internal” or “abdominal”) inguinal ring and the superficial (also called the “external” or “subcutaneous”) inguinal ring, respectively. These are described in more detail below in relation to FIGS. 1-4D.

When the body is in an upright orientation, the inguinal canal B (see FIGS. 1 and 4B) has an anterior and a posterior wall and a roof and a floor. The anterior wall is nearest to the skin, and is formed by the aponeurosis 1 (layers of flat, broad tendons) of the external oblique muscle, and by the internal oblique muscle 2 for its outer third and sometimes even its outer half. The posterior wall 3 is nearest to the vertebral column. It is formed by the fascia of the transversalis (a muscle layer of the anterior and lateral abdominal wall) and at its inner third the conjoined tendon. The roof 4, nearest to the head, is formed by the arching fibers of the internal oblique muscle and (still farther above) the transversalis. The floor is nearest to the feet.

The deep inguinal ring 5, which forms the entrance to the inguinal canal B, is a slit in the transverse fascia that is located about 1 cm above and lateral to midpoint of the inguinal ligament 7 (see FIG. 1). The superficial inguinal ring 6 (see FIG. 4A), which forms the exit from the inguinal canal, is a triangular shaped slit above and just lateral to the pubic tubercle. This ring is an opening in the external oblique aponeurosis.

The inguinal canal transmits the vas deferens (spermatic cord) 8 (FIG. 1) in the male and the round ligament in the female. The inguinal canal passes obliquely through the abdominal wall C (FIG. 4B) above the inguinal ligament. The inguinal ligament 7 is formed by the aponeurotic fibers of the external oblique muscle, and stretches from the anterior superior iliac spine to the pubic tubercle. The iliopsoas muscles, the femoral vein artery and nerve pass below the inguinal ligament 7.

The deep inguinal ring 5 is the entrance to the inguinal canal on the inside of the abdominal wall. As the canal passes through the abdominal wall C, it receives a layer of muscle from the internal oblique, the cremaster muscle. At the superficial inguinal ring 6 the inguinal canal passes through the external oblique aponeurosis and receives a layer from the aponeurosis, the external spermatic fascia in the male. The deep inguinal ring lies laterally to the inferior epigastric vessels. The superficial inguinal ring lies above and medial to the pubic tubercle.

There are two types of inguinal hernias: direct E (FIG. 4D) and indirect F (FIG. 4C). A direct inguinal hernia protrudes through the superficial inguinal ring 6, directly through the abdominal wall as shown schematically in FIG. 4D. An indirect inguinal hernia protrudes through the deep inguinal ring 5, going through the inguinal canal, pushing on structures within, and emerging through the superficial inguinal ring 6 and attaches to the spermatic cord as shown in FIG. 4C.

A conventional operation to repair hernias was developed by Bassini and is commonly referred to by the Bassini name. In this procedure, the neck of the hernia sac is exposed by incising the aponeurosis of the external oblique, and then the spermatic cord 8 (FIG. 1) is separated from it. The hernia sac is to be replaced and ligated as high as possible and cut away. The spermatic cord 8 is then raised and the arching fibers of the internal oblique (and transversalis) are sutured beneath it to the inguinal ligament. The spermatic cord is then replaced, and the cut edges of the external oblique are sewn together down to the external ring, leaving sufficient room for the exit of the cord (See FIG. 3). It is also known to add surgical mesh between the inguinal canal floor and the spermatic cord, typically by numerous sutures to attach the mesh to the floor all around the circumference of the mesh.

The conventional procedure for repairing inguinal hernias does not provide the maximum level of protection from future hernias, nor do the conventional structures used to repair a hernia.

### BRIEF SUMMARY OF THE INVENTION

A device and method have been developed for surgical repair of both direct and indirect inguinal hernias. One contemplated embodiment of the system described above includes a plug and an onlay mesh patch that are not coated. In this embodiment, an onlay mesh patch is a generally fabric-like sheet of mesh material that is designed to be compatible with human tissue, thus making it a surgical mesh. The patch has a large area with edges, and one end has a void defining two legs. The plug mounts to one face of the patch near the proximal end of one leg. The plug can be integral with the patch, but is contemplated to be mounted to the patch by sufficient means, including a suture or staple.

Another embodiment of the system uses a very similar patch and attached mesh plug coated with polylactic acid, polyglycolic acid, polyethylene glycol (PEG), or collagen on the mesh onlay patch, the plug, or both the patch and the plug. All or some of the contemplated systems' components are coated with one or more of these substances. These substances promote a reaction by the patient's body that tends to encourage inflammation of the contacting tissue and more rapid growth of the tissue into the mesh, the plug or both. The resulting scarring is also advantageous, and is enhanced by one or more of these material coatings.

The mesh system described herein is utilized for repair of both direct and indirect inguinal hernias. When installed to repair one type of hernia, the system also prevents, or at least reduces the probability of, the other type of hernia from occurring subsequently. Thus, by utilizing the system for a direct inguinal hernia repair, the system assists in the repair of the direct inguinal hernia via the onlay mesh. The attached mesh plug placed in the internal ring at the time of the procedure prevents or reduces future indirect inguinal hernias. Likewise, whereas the mesh plug at the internal ring is utilized for repairing the indirect inguinal hernia, the attached onlay mesh patch prevents or reduces the occurrence of direct inguinal hernias. The extended coverage provided by the onlay mesh patch also assists in preventing the recurrence of direct inguinal hernias at the pubic tubercle region and inguinal hernias occurring lateral to the internal ring.

The system described herein is advantageous due to pre-surgical attachment of the onlay patch and plug mesh. This permits the surgeon to attach the entire system by inserting a pre-compressed plug into the deep inguinal ring, and then expanding the plug by removal of the holder. The holder can be a Kelly clamp or similar, and then sutures or other attachments to a few select locations hold the system in place. The larger size of the onlay mesh patch reduces subsequent hernias, and the growth material applied to the mesh enhances the growth of the tissue surrounding the mesh into the mesh. A method of attaching the system and a combination of the system and the human body in which the system is applied also have substantial advantages.

### BRIEF DESCRIPTION OF THE SEVERAL VIEWS OF THE DRAWINGS

FIG. 1 is a front cut-away view illustrating a human inguinal canal exposing the spermatic cord among other structures.

FIG. 2 is a front cut-away view illustrating the inguinal canal of FIG. 1 with covering structures shown prior to full exposure as in FIG. 1.

FIG. 3 is a front cut-away view illustrating the inguinal canal of FIG. 1 after a portion of a conventional hernia repair.

FIG. 4A is a schematic front view illustrating the general location of the inguinal canal on a human male, and the general locations of the deep and superficial rings.

FIG. 4B is a schematic view in section illustrating the inguinal canal and its relation to the abdominal wall.

FIG. 4C is a schematic view in section illustrating the view of FIG. 4B showing the general location of an indirect inguinal hernia.

FIG. 4D is a schematic view in section illustrating the view of FIG. 4B showing the general location of a direct inguinal hernia.

FIG. 5 is a top view illustrating the preferred embodiment of the mesh patch and plug system.

FIG. 6 is a side view in section illustrating the preferred embodiment of the mesh patch and plug system.

FIG. 7 is a front cut-away view illustrating a human inguinal canal exposing the spermatic cord and examples of the two types of inguinal hernias, among other structures.

FIG. 8 is a front cut-away view illustrating the human inguinal canal of FIG. 7 with the direct hernia partially repaired and an indirect hernia sac still protruding from the deep inguinal ring.

FIG. 9 is a front cut-away view illustrating the human inguinal canal of FIG. 8 after the direct hernia has been repaired by a running Bassini technique closure and the indirect hernia has been replaced through the deep inguinal ring.

FIG. 10A is a front cut-away view illustrating the human inguinal canal of FIG. 9 after a running Bassini technique closure has been performed.

FIG. 10B is a side view in section showing the mesh patch and plug system with a Kelly clamp holding the plug in a compressed state through the onlay patch.

FIG. 11 is a is a front cut-away view illustrating the human inguinal canal of FIG. 10A after the mesh patch plug system has been inserted beneath the spermatic cord and the compressed plug has been inserted into the deep inguinal ring.

FIG. 12 is a front cut-away view illustrating the human inguinal canal of FIG. 11 after the legs of the onlay patch have been connected around the spermatic cord and the mesh plug has been sutured to the fascia near the deep inguinal ring.

FIG. 13 is a front cut-away view illustrating the human inguinal canal of FIG. 12 after the attachments to the tissue in the floor of the inguinal canal have been made.

FIG. 14 is a front cut-away view illustrating the human inguinal canal of FIG. 13 after closure of the surgical site has been performed.

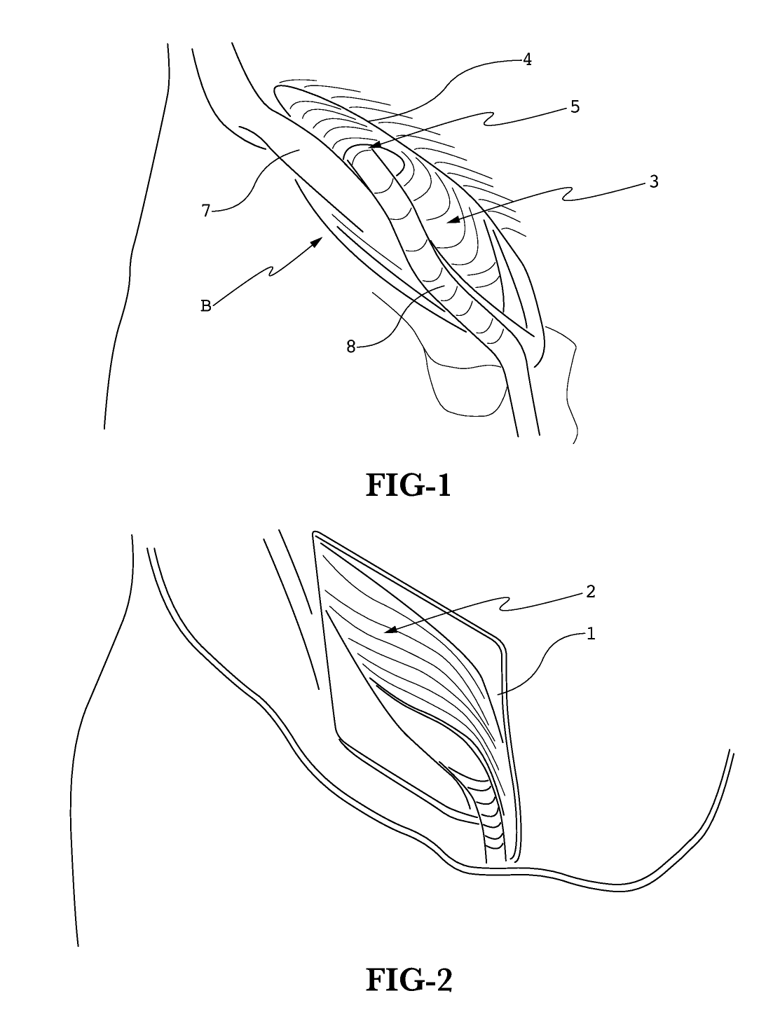

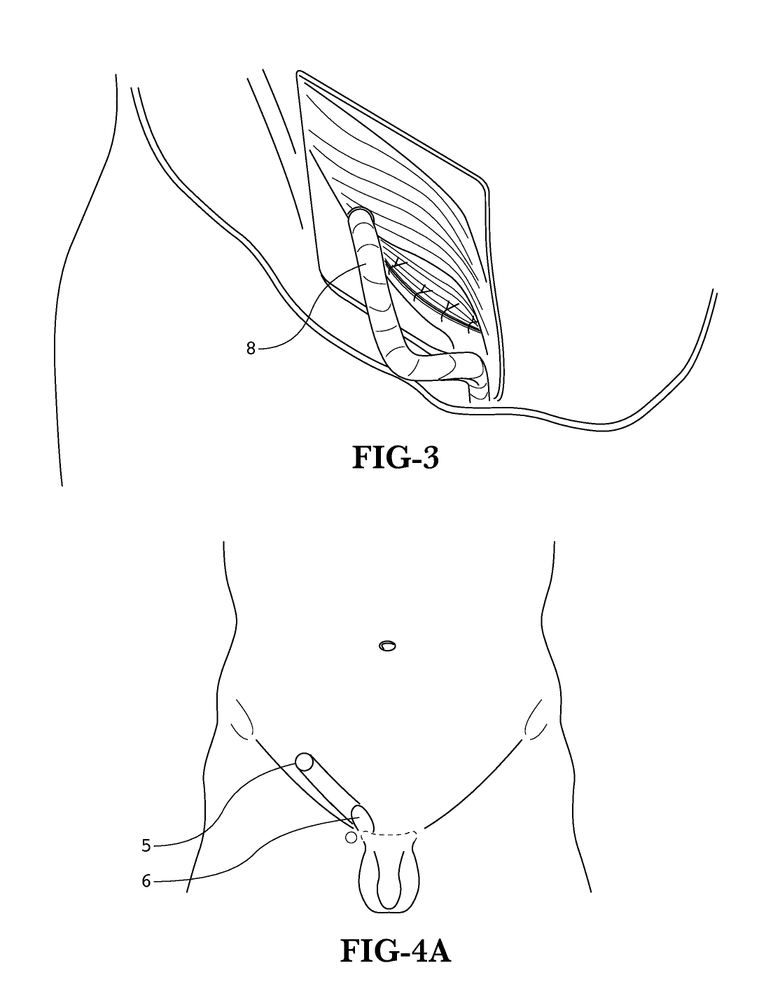

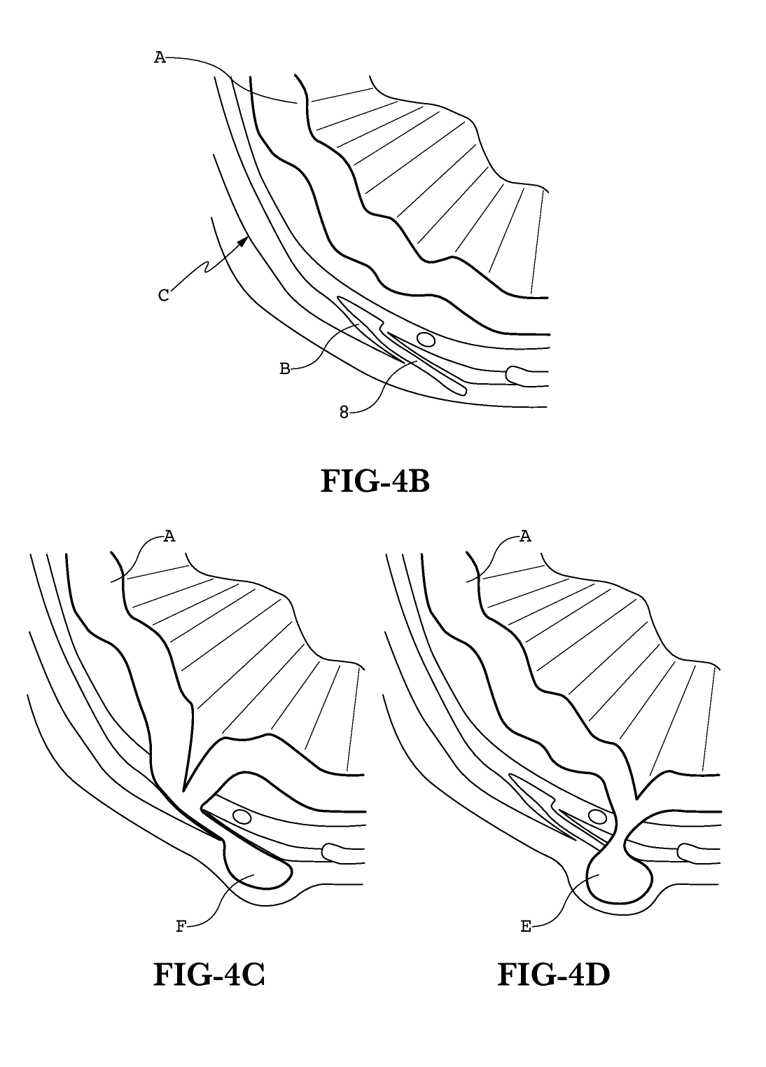

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or terms similar thereto are often used. They are not limited to direct connection, but include connection through other elements where such connection is recognized as being equivalent by those skilled in the art.

### DETAILED DESCRIPTION OF THE INVENTION

Patent application Ser. No. 61/288,432 is incorporated in this application by reference.

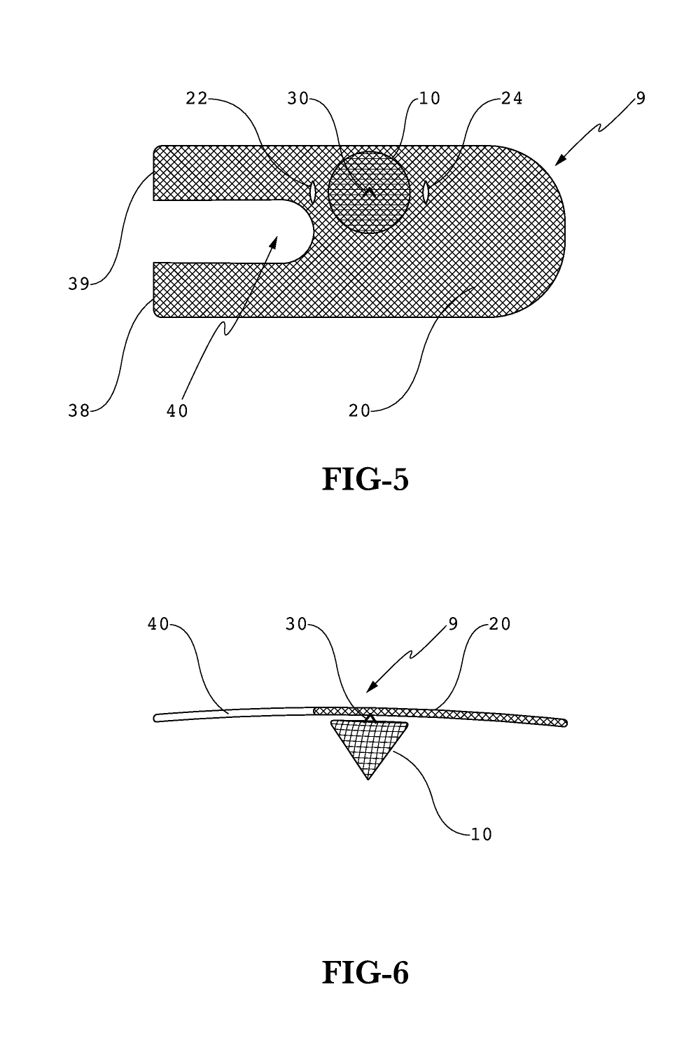

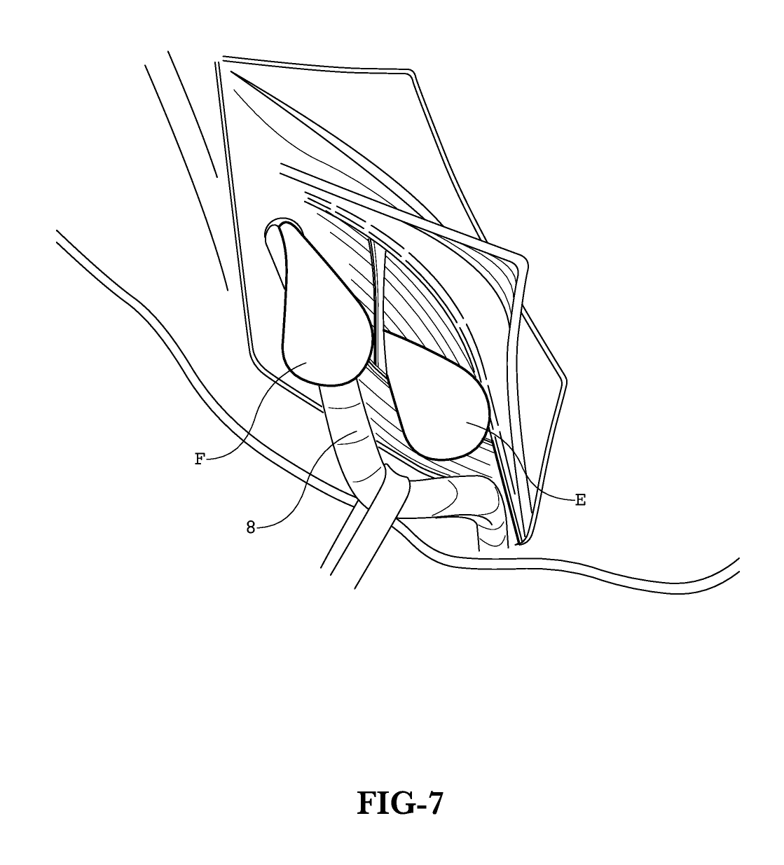

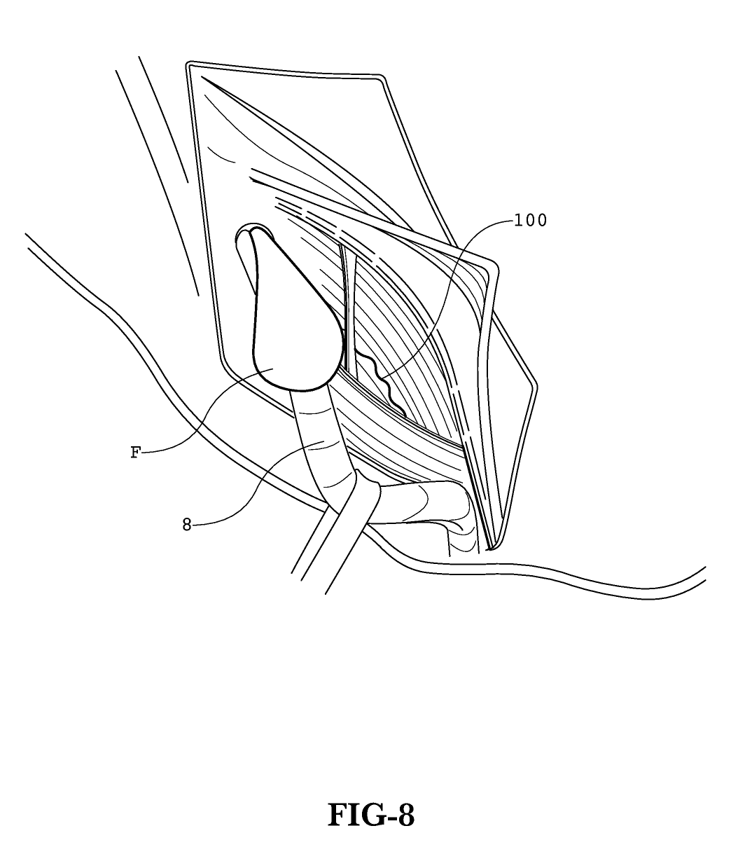

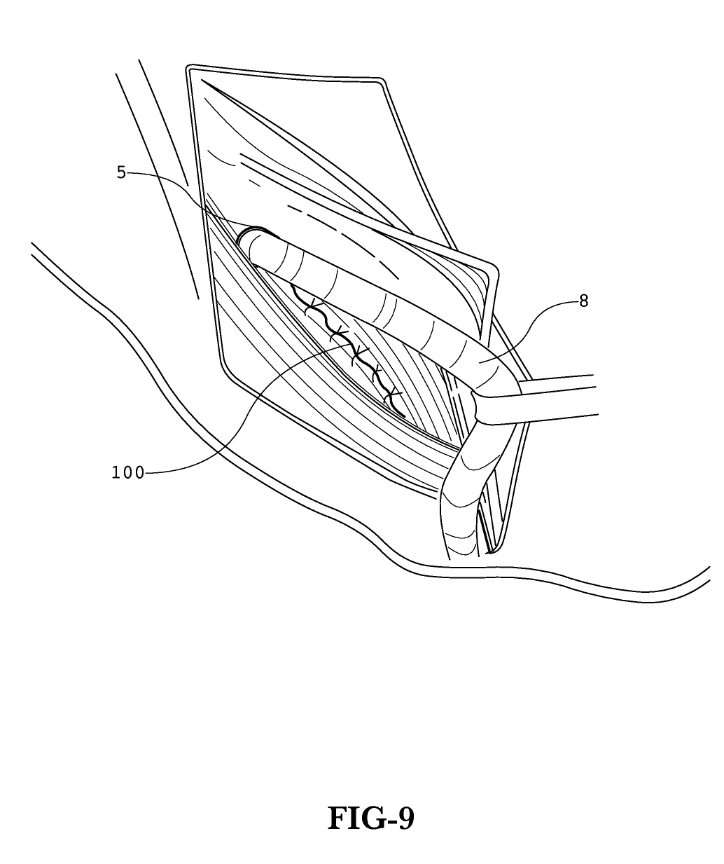

A combination mesh patch and plug system 9 is shown in FIGS. 5 and 6. A first component of this system 9 is a mesh plug 10 and a second component is a mesh patch 20 as illustrated in FIG. 5 from the top and FIG. 6 from the side. The plug 10 and patch 20 are preferably made of a surgical Prolene mesh material, similar to that sold by medical suppliers for conventional hernia repairs. Prolene is a synthetic, monofilament, non-absorbable polypropylene suture material that can be woven to form a mesh material having fibers oriented transverse to one another in a conventional, two-dimensional fabric. The Prolene mesh is preferably a fabric with openings between fibers that are substantial, such as between a fraction of a millimeter to a few millimeters. Of course, any similar, suitable material can be substituted for the preferred Prolene mesh, as will be understood by a person of ordinary skill from the disclosure herein. The term “surgical mesh” refers to a class of flexible sheets that permit the growth of tissue through openings in the mesh after the surgery has been completed to enhance attachment to surrounding tissue. Other materials that do not have a “mesh” configuration may be designed to provide similar attachment to surrounding tissue. Nevertheless, such materials will be known by the person having ordinary skill to be a substitute for “surgical mesh”, and are considered the same as a surgical mesh.

The plug 10 is formed in a conical shape, as shown in FIG. 6, by taking a strip of Prolene mesh and winding it around a central axis. Alternatively, the plug 10 can be formed by a plurality of mesh cones of slightly different size nesting in one another. A modified conventional plug Prolene mesh is also contemplated. Although the plug 10 is shown and described as being conical, it is contemplated that any tapered shape will suffice to permit the plug 10 to be used in accordance with the invention. The conical plug 10 is anchored at its flat end to the patch 20 as shown in FIG. 6 using a Prolene anchoring stitch 30 or a clip, staple or any other suitable attachment prior to the initiation of the surgical procedure. This mechanical attachment of the plug 10 to the patch 20 combines the components of the system 9 into a unitary structure unknown to the inventor in the field of onlay mesh patches and plug meshes.

The onlay mesh patch 20 is preferably a generally oval-shaped, pliable onlay Prolene mesh. Two slots 22 and 24, which are significantly larger openings through the plane of the patch 20 than the openings throughout the mesh, are formed directly above the Prolene mesh plug 10 at about the three o'clock and nine o'clock positions around the generally circular region where the end of the plug 10 contacts the patch 20 (see FIG. 5). The slots 22 and 24 allow the tips of a Kelly clamp (or equivalent) to pass easily through the plane of the patch 20 and thereby seat against opposite sides of the crimped and compressed mesh plug 10 on the underside of the patch 20. The compression occurs when placing the mesh plug 10 at the desired position in the internal ring as described in more detail below. The anchoring stitch 30 is loose enough to allow for some mobility of the plug 10 relative to the patch 20, thus preventing postoperative neuralgias. The slots 22 and 24 may be unnecessary if the openings in the mesh permit the instrument to pass through.

Prior to the surgery, the Prolene plug 10 is directly attached to the onlay Prolene mesh 20 via anchoring Prolene stitch 30 or clip attachment, preferably by a manufacturing process. Thus, the system shown in FIGS. 5 and 6 is preferably ready to use prior to the surgical procedure being initiated. The system 9 is thus devised as a single unit consisting of the patch 20 and the plug 10 attached to the patch 20 via anchoring attachment and preferably contained together in a sterile package.

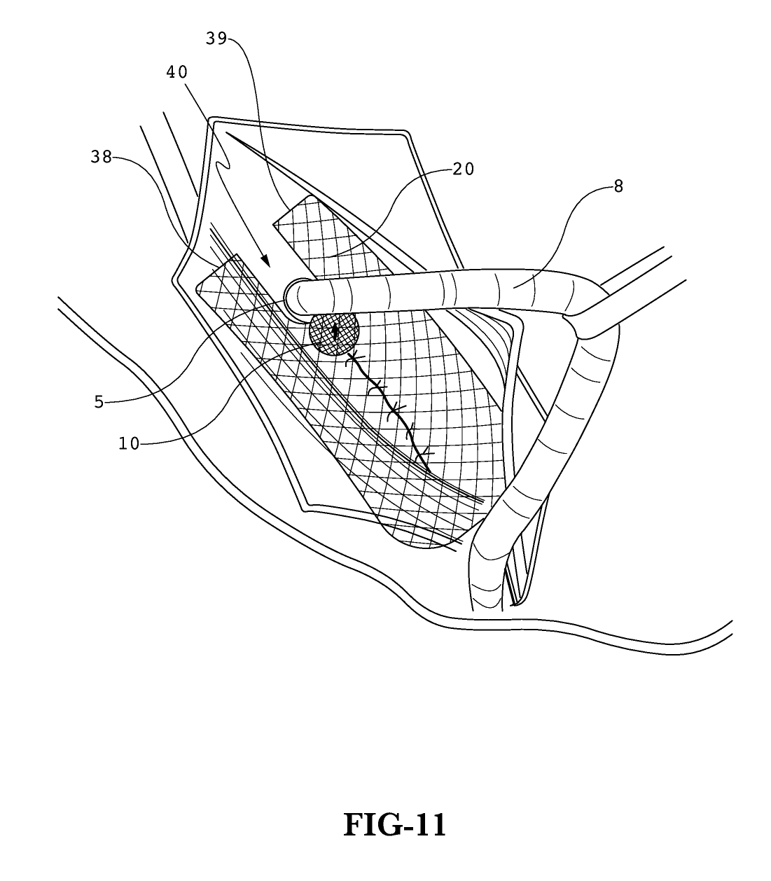

As shown in FIG. 5, the onlay mesh patch 20 has an elongated “keyhole defect” 40 through which the spermatic cord can extend (as shown in FIG. 11) to lie over the mesh patch 20. This allows the onlay mesh patch 20 to have direct apposition with the floor of the inguinal canal (see FIG. 11). The keyhole defect 40 may be further cut and shaped, if needed, during the operation per the surgeon's preference to allow the spermatic cord to fit appropriately in the keyhole defect 40. Likewise, the rest of the mesh patch 20 can be cut to the desired shape and size.

An important feature of the onlay mesh patch 20 is that it has outer edges that extend about 3 cm circumferentially in all directions above the inguinal canal region. This assists in the appropriate postoperative scarring between the patient's native tissue and the mesh, thereby requiring only minimal sutures to be utilized for securing the onlay mesh patch. Minimal absorbable sutures are utilized during the repair, thus assisting in preventing postoperative neuralgias. The anchoring stitches for the mesh system 9, which are described in more detail below, are preferably absorbable 2-0 Vicryl sutures that are tied loosely so as to prevent the possibility of nerve entrapment/neuralgias postoperatively.

In summary, the system 9 is utilized for the repair of either or both types of inguinal hernia (direct or indirect). When one type of inguinal hernia is repaired, the system 9 has the secondary effect of preventing the development of the other type of inguinal hernia, in addition to preventing the development of the same type of inguinal hernia that was repaired.

### Description of a Surgical Procedure Using the Device

The open inguinal hernia repair procedure, preferably performed using the system 9 described above, is preferably carried out as follows. The patient is taken to the operating room and placed under anesthesia of choice. An ilioinguinal nerve block is preferably performed after the patient is prepped and draped appropriately. The nerve block, prepping and draping is conventional.

A small incision is made in the inguinal region and the external oblique aponeurosis is identified and opened appropriately. Exposure of the inguinal canal is assisted with use of a wheat Lander retractor. The external oblique aponeurosis is nicked with a #15 blade at the pubic tubercle region and then opened with Metzenbaum scissors laterally and medially. The edges of the cut external oblique aponeurosis are exposed superiorly and inferiorly with use of Allis clamps or hemostats.

The spermatic cord and ilioinguinal nerve are exposed with a gentle finger sweep and elevated out of the field with a Penrose drain. The ilioinguinal nerve is identified and included with the spermatic cord within the Penrose drain if possible. In the following description, both a direct and an indirect hernia are repaired simultaneously intraoperatively, i.e., a Pantaloon hernia. Of course, the person having skill in the surgical fields will be able to determine, from the description herein, how to perform the repairs separately.

For an indirect hernia, the indirect sac is identified and gently pulled away from the spermatic cord. The indirect sac may be opened, inspected, and highly ligated. Alternatively, the indirect hernia sac may be separated from the spermatic cord and placed back within the deep inguinal ring. FIG. 9 shows the surgical site after placement of the indirect sac back into the deep inguinal ring.

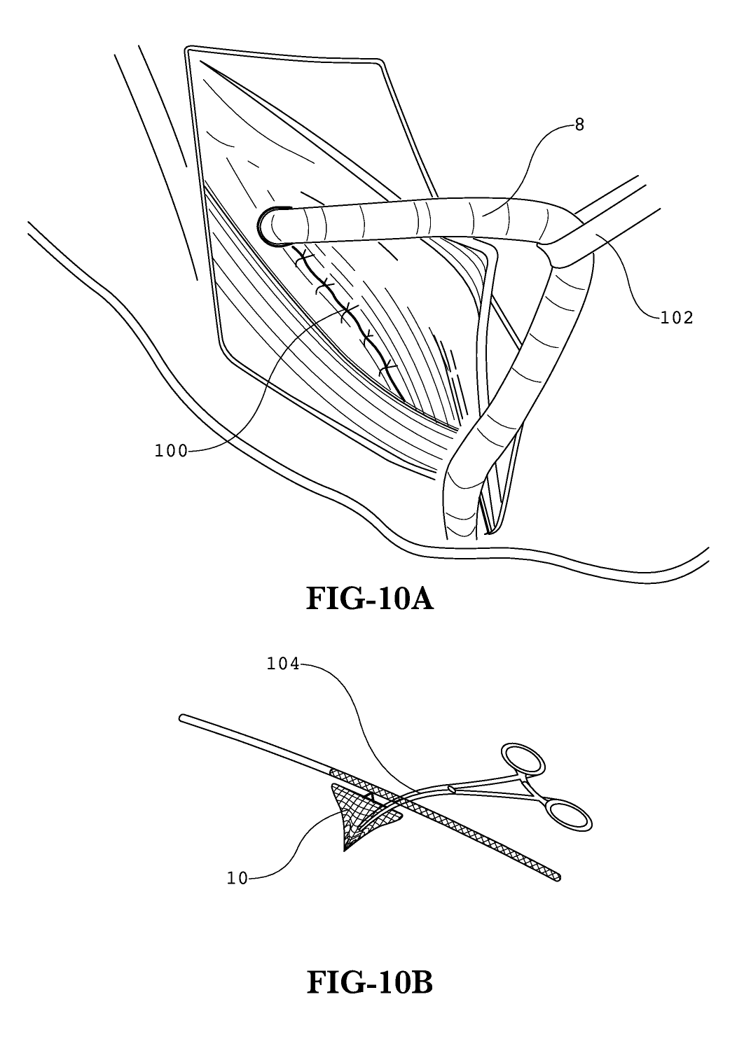

For a direct inguinal hernia, and with reference to FIGS. 9 and 10A, the defect in the inguinal canal floor is closed via a modified running Bassini technique closure 100 with permanent suture of the surgeon's choice. With utilization of the system 9, the inguinal canal floor is first closed via the modified running Bassini technique closure 100 with a permanent suture for both indirect and direct inguinal hernias. The deep inguinal ring 5 can then be tightened, but not completely closed, with the closure 100 sufficient to allow easy passage of a closed Kelly clamp prior to the placement of the mesh plug 10 as described below. By tightening the deep inguinal ring somewhat, but not completely, the mesh plug 10 has a snug fit when placed through the deep inguinal ring, and only minimal absorbable sutures are required for appropriately securing the plug 10 in the deep inguinal ring.

In FIG. 10A conventional structures, such as Allis clamps (not shown), hold back the external oblique aponeuroses. After the hernias are repaired as described above, the spermatic cord 8 is elevated with a Penrose drain 102 and the patch and plug system is placed under the spermatic cord 8.

Preferably the system 9 is displaced from a caudal to cephalid direction, into the desired position with a Kelly clamp holding the system 9 to the position shown in FIG. 11. Conversely, the patch/plug system 9 may be placed under the spermatic cord from a cephalid to caudal direction, depending on the patient's anatomy and habitus determined intra-operatively.

Prior to positioning the system 9 in the inguinal canal, a Kelly clamp, or a similar device with slender tips that can extend through slots 22 and 24 in the onlay patch 20, holds the system 9 as follows. First, the surgeon compresses the mesh plug 10 manually using his or her thumb and index finger of a first hand, such as by twisting, rolling and/or compressing the material of the mesh plug 10. The purpose of this step is to take advantage of the inherent elasticity of the mesh plug 10 by making the plug 10 smaller than when it is in a relaxed position so that, upon release of the compressive force, the plug 10 expands and exerts a radially-outwardly directed force against any surface the plug 10 encounters prior to full expansion. As shown in FIG. 10B, the tips of the Kelly clamp 104 are then placed through the slots 22 and 24 of the onlay mesh 20 on opposite sides of the mesh plug 10 and drawn together by manually squeezing the handles of the clamp in a conventional manner, preferably with the surgeon's other hand (not shown). This provides a clamping force on opposite sides of the plug 10 to keep the mesh plug 10 in, or close to, its desired compressed state prior to placement within the deep inguinal ring. The mesh plug 10 is then held in position with the Kelly clamp 104 closed around, and clamping, the mesh plug 10. Thus, the surgeon holds the entire system 9 with the Kelly clamp 104 as shown in FIG. 10B with the plug 10 shown compressed. The degree of compression illustrated is but one example, and it will be understood that the plug 10 can be compressed more or less than shown in FIG. 10B.

Of course, an assistant to the surgeon can compress the plug 10 and clamp it with the Kelly clamp, or the system 9 can come pre-compressed with or without a Kelly clamp holding the plug 10 in the compressed condition. For example, a surgical suture loop or equivalent holding structure can be placed over or around the compressed plug before the surgery, such as during manufacture, to hold the plug in the compressed state prior to the start of the surgery. Then the surgeon can remove the holding structure from the plug prior to closing the surgical site, whereupon expansion would occur. In such a case, the need for slots around the attachment site of the plug would be eliminated. However, a different opening for insertion of a knife blade, scissors or other instrument, is contemplated in order to enable the surgeon to sever or otherwise remove the holding structure from the plug to release the compression of the plug. The holding structure can be incorporated into the plug so that the holding structure can remain in the patient, or it can be removed entirely.

With the spermatic cord elevated with the Penrose drain 102 as shown in FIG. 10A and the system 9 held by the Kelly clamp as shown in FIG. 10B, the system 9 is brought under the spermatic cord (FIG. 11) with the spermatic cord 8 extending through the keyhole defect 40 near the deep inguinal ring 5. The pointed end of the conical mesh plug 10 is inserted into the deep inguinal ring 5 until the sidewalls of the plug 10 seat against the sides of the deep inguinal ring. Once the sidewalls seat, the Kelly clamp (or other structure holding the plug 10 in the compressed state) is removed from the mesh plug 10, thereby permitting the previously compressed mesh plug 10 to expand due to the elastic “memory” of the mesh material of which the plug 10 is made. FIG. 11 illustrates the system 9 in position after removal of the Kelly clamp.

As noted above, prior to placement of the mesh plug 10 in the deep inguinal ring, the surgeon compressed the mesh plug 10 and held it with the Kelly clamp. This allowed placement of the smaller mesh plug 10 within the deep inguinal ring followed by subsequent expansion of the plug 10 in the deep inguinal ring by release of the Kelly clamp. The expansion of the plug 10 against the walls of the deep inguinal ring acts, at least in part, to retain the plug 10 in place.

It should be noted that closure of the floor of the inguinal canal with the modified running Bassini technique and tightening of the deep inguinal ring creates a “shutter valve” effect at the deep inguinal ring to assist in anchoring the mesh plug 10 after its self-expansion. In order to determine how much to tighten the deep inguinal ring, one must be able to assure the easy passage of a closed Kelly clamp (or similarly sized structure) into the deep inguinal ring once the floor of the inguinal canal has been closed with the modified running Bassini technique closure. If the deep inguinal ring has been closed too tightly, it will be difficult to place the mesh plug 10 in its desired position. Therefore, prior to placing the system 9 beneath the spermatic cord 8, one should test the tightness of the deep inguinal ring 5 to ensure that the compressed mesh plug 10 can be inserted therein.

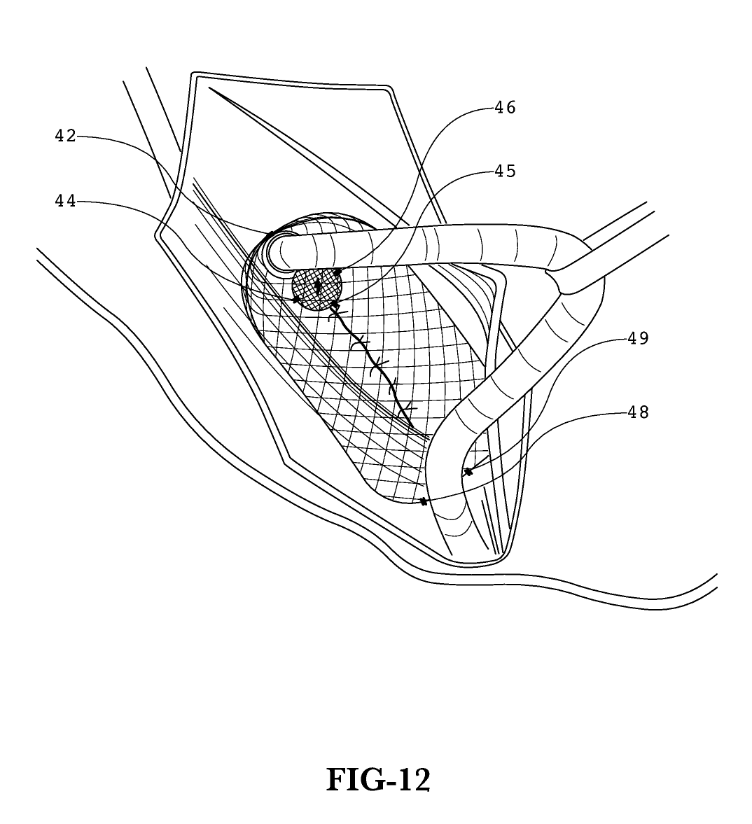

Once the mesh plug 10 is in position in the deep inguinal ring, the two legs 38 and 39 of the onlay mesh patch 20 at opposite sides of the keyhole defect 40 are brought around the spermatic cord appropriately until they contact one another as shown in FIG. 12. The legs 38 and 39 of the mesh onlay 20 are then secured together, preferably using 2-0 Vicryl suture 42, but alternatively using surgical tacks, snaps, specialty fasteners or equivalent attaching means. Care must be taken not to suture the legs 38 and 39 of the mesh patch too tightly around the spermatic cord 8. A closed Kelly clamp should be able to pass between the spermatic cord and the edge of the patch 20 at the keyhole defect 40 once the legs 38 and 39 have been secured together as shown in FIG. 12. Of course, other gauges can be used to determine whether the legs 38 and 39 are too tight around the cord 8.

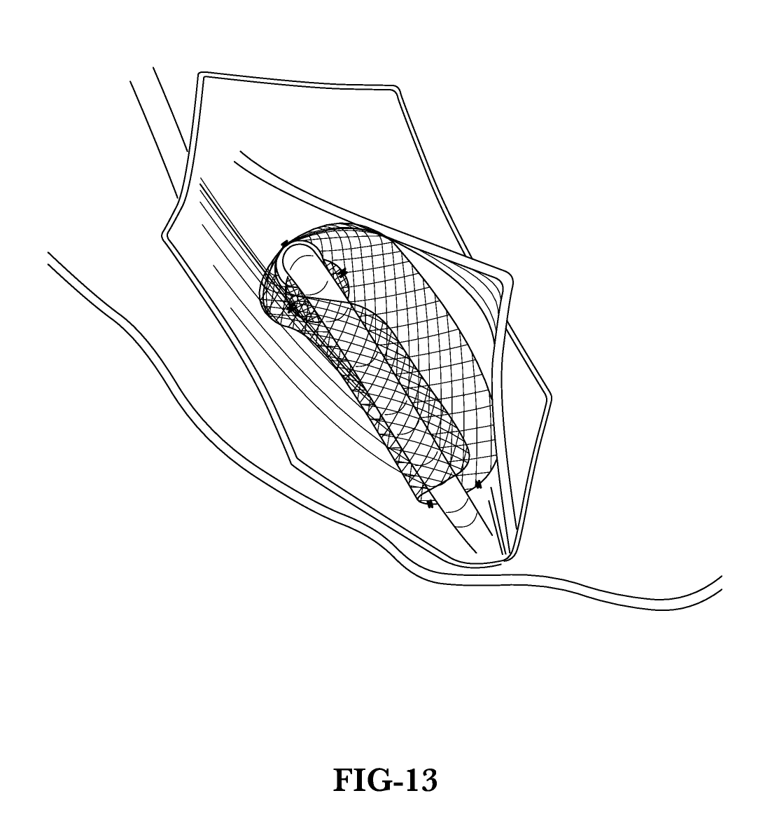

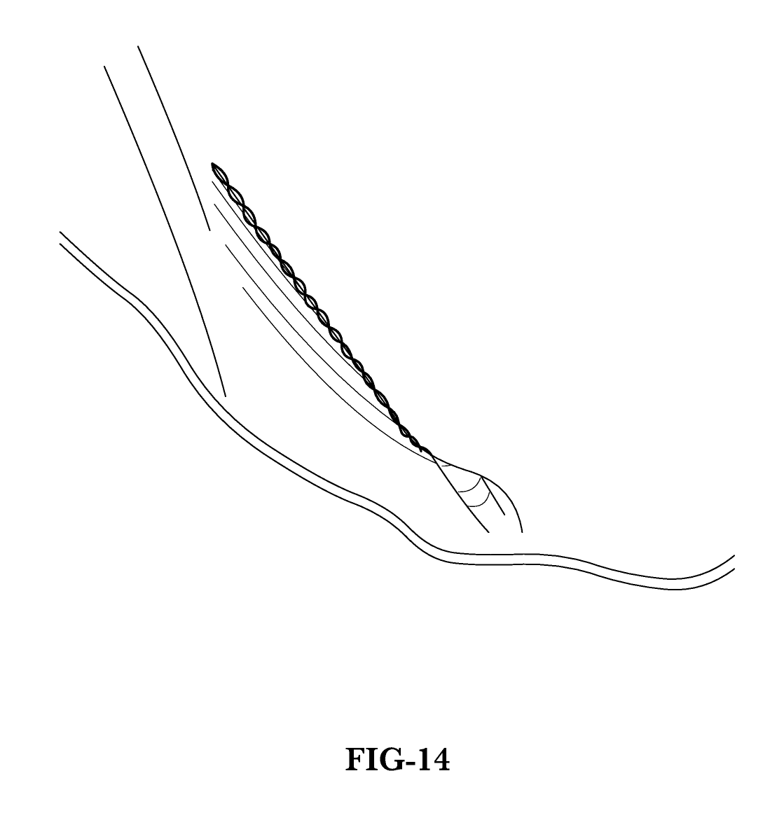

The onlay mesh patch 20 is then placed under the external oblique superior, and also lateral, to the spermatic cord. Care is taken not to damage the iliohypogastric nerve. Direct visualization of the iliohypogastric nerve is assisted with the use of an Allis clamp or hemostat that has been described during the earlier steps of the procedure. A gentle finger sweep can assure that there is sufficient space for the mesh patch 20 to have a nice lie under the external oblique superiorly and laterally to the deep inguinal ring. The onlay mesh patch 20 is also placed medially at the pubic tubercle region and inferiorly under the spermatic cord. Good exposure at the pubic tubercle region can be obtained with the use of a Goulet or a Richardson retractor by elevating the spermatic cord medially so as to expose the pubic tubercle region. The onlay mesh patch 20 extends about 3 cm under the external oblique aponeurosis superiorly, about 3 cm lateral to the spermatic cord and deep inguinal ring, about 3 cm over the pubic tubercle region, and about 3 cm above the ilioinguinal ligament. See FIG. 13. One edge of the onlay mesh patch 20 wraps around the spermatic cord inferiorly at the ilioinguinal ligament in the inguinal canal region as what is described as a “cigarette type” wrapping fashion of the mesh around the spermatic cord, and this wrapping action typically occurs at, or just before, the closure of the tissue over the opened inguinal canal, as noted below.

Once the system 9 is placed under the spermatic cord, both the onlay mesh 20 and attached plug mesh 10 are attached at the medial deep inguinal ring with, for example, three stitches 44, 45 and 46, preferably using 2-0 Vicryl suture or equivalent, and preferably located approximately evenly around the perimeter of the approximately circular end of the plug 10—for example, at about every 90 degrees. The absorbable suture incorporates both the onlay mesh patch 20 and the attached mesh plug 10 for anchoring the system 9 to the tissue at the deep inguinal ring medially. Good exposure of the deep inguinal ring is assisted with the use of a vein retractor. Diligent care must be taken throughout the entire surgery to identify all the nerves when placing the absorbable anchor sutures.

After attachment of the plug 10 and patch 20 to the region around the deep inguinal ring, the onlay mesh patch 20 is attached to the fascia of the pubic tubercle region with, for example, two stitches 48 and 49, preferably using 2-0 Vicryl suture. This attachment can be about 2 cm proximal to the pubic tubercle. Care must be taken not to incorporate the suture into the pubic tubercle bone so as to prevent postoperative neuralgias. The use of absorbable sutures will assist in preventing neuralgias once the mesh has been incorporated into the native tissue and the suture dissolves. Of course, this, along with the sutures placed at the deep inguinal ring, can be replaced by surgical tacks or any other equivalent means for attaching the structures to the surrounding tissue.

Once these two attachments are in place, the mesh patch 20 can also be anchored superiorly at the internal oblique muscle with, for example, one stitch, and/or inferiorly at the ilioinguinal ligament with, for example, one stitch (not shown). Both anchors preferably use 2-0 Vicryl suture or equivalent anchoring means, such as a surgical tack, if needed to obtain a nice lie of the mesh within the inguinal canal region.

The first knot of all sutures is preferably loosely tied with an air knot so as to assist in preventing postoperative neuralgias if a nerve is inadvertently entrapped by the surgeon. It should be understood that the anchor sutures at the pubic tubercle region may be made initially followed by the anchoring sutures at the deep inguinal ring, followed by the closure of the keyhole defect 40. Of course, the order of these steps can be varied based upon the surgeon's judgment of the best order. The order of these steps is described as preferred, but the surgeon is able to modify the order depending on the circumstances. Regardless of the order, it is desirable to have a nice lie of the mesh in all directions once all the anchoring sutures have been placed.

Once the procedure has progressed to this point, the mesh patch 20 is inspected for a nice lie within the inguinal canal region. By assuring that the edges of the mesh patch 20 that have been secured have been placed under the external oblique laterally and superiorly with a gentle finger sweep, these edges of the mesh will not fold toward the spermatic cord and will have a nice lie in all directions. On the other hand, the excess mesh at the inguinal canal region is allowed to wrap around the spermatic cord as shown in FIG. 13, thus allowing the mesh to directly abut the inguinal ligament and aid in postoperative scar formation. By incorporating the ilioinguinal nerve with the spermatic cord at the initial exposure with the Penrose drain, nerve entrapment will be reduced when the mesh scars in with the ilioinguinal ligament. All nerves are inspected again prior to closure. Once again, the iliohypogastric nerve is inspected to verify the mesh has a nice lie under the external oblique aponeurosis superiorly and that there is no entrapment of the nerve.

The onlay mesh 20 preferably extends about 3 cm in all directions beyond a conventional mesh used for hernia repairs, which reduces the probability of a direct hernia occurring or recurring at the pubic tubercle, or a hernia lateral to the deep inguinal ring. By extending the coverage of the onlay mesh patch 20, the patient's native, inherent Valsalva maneuver will also assist in keeping the system 9 in the desired position postoperatively. When the PEG or other coating is applied, this inflames the tissue and enhances growth of the tissue into the onlay mesh and plug.

Good closure of the external oblique aponeurosis is desirable, of course. This assists in keeping the system 9 in the desired position postoperatively. It will also assist in the postoperative scarring that will occur between the patient's own native tissue and the onlay

Prolene mesh patch 20.

A circulator pulls the testicle back within the scrotum. The external oblique aponeurosis is then closed using a running 2-0 Vicryl suture as shown in FIG. 14. The subcutaneous and Scarpa's tissues are closed, preferably using running 2-0 chromic suture in a double layered manner. The skin is surgically stapled and an occlusive dressing is placed, thus completing the inguinal hernia repair.

The same basic concepts described above for males can be utilized for inguinal hernia repairs in females with the exception that the round ligament is preferably removed and sent to the pathologist. Additional anchoring sutures may also be required to assure that the mesh has a nice lie in the inguinal canal region with female inguinal hernia repairs. These sutures can be superior and inferior and attached to the floor. Furthermore, it should be noted that the exact number of stitches is not required, nor are more stitches prohibited. Indeed, although it is generally desirable to keep the number of stitches low to reduce trauma and the probability of later complications, the surgeon has the ability to modify the number and placement of stitches as he or she sees fit for the circumstances. The preferred number and placement noted herein is preferred for the typical situation in the inventor's experience.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. A hernia repair system comprising:
(a) an elongated, substantially planar surgical mesh patch having first and second opposing faces, first and second opposing edges and first and second opposing ends;
(b) first and second spaced legs formed in the first end of the patch and defined by an elongated gap in the patch therebetween; and
(c) an elastic, compressible conical plug having a first end that is tapered and a second, opposing end that is substantially planar, the second end of the plug mounted to the first face of the patch.

2. The hernia repair system in accordance with claim 1, wherein the conical plug is held in a pre-compressed state by a holding structure that is configured to release at least some of the pre-compression during a surgical procedure in which the system is implanted in a live human.

3. The hernia repair system in accordance with claim 2, further comprising a coating on the mesh patch to promote incorporation of the mesh patch into contacting human tissue.

4. The hernia repair system in accordance with claim 2, wherein the conical plug is formed from a plurality of nested, conically shaped pieces of surgical mesh.

5. A method of installing a hernia repair system, the method comprising:
(a) disposing an elongated, substantially planar surgical mesh patch between a human's inguinal canal floor and spermatic cord, the patch having first and second opposing faces, first and second opposing edges and first and second opposing ends with first and second spaced legs formed in the first end of the patch and defined by an elongated gap therebetween;
(b) disposing the spermatic cord between the legs of the patch;
(c) attaching the first leg to the second leg on an opposite side of the spermatic cord as the second end of the patch;
(d) extending a tapered first end of a pre-compressed, conical plug into a deep inguinal ring of the human, the second, opposing end of the plug being substantially planar and mounted to the first face of the patch;
(e) releasing the pre-compression of the conical plug, thereby permitting the conical plug to expand within the inguinal ring;
(f) attaching the patch and the plug to fascia in the floor of the inguinal canal;
(g) wrapping at least a portion of the patch around the spermatic cord; and
(h) closing the surgical site.

6. The method in accordance with claim 5, further comprising pre-compressing the conical plug prior to any step of claim 5, and holding the pre-compression.

7. The method in accordance with claim 5, further comprising attaching the patch to the human at the soft tissue near the human's pubic tubercle.

8. A hernia repair system in combination with a human body, the combination comprising:
(a) an elongated, substantially planar surgical mesh patch having first and second opposing faces, first and second opposing edges and first and second opposing ends, the patch disposed between a human's inguinal canal floor and spermatic cord with at least a portion of the first face seated against an inguinal canal floor;
(b) first and second spaced legs formed in the first end of the patch and defined by an elongated gap in the patch therebetween, the spermatic cord extending through the elongated gap, the first leg extending around one side of the spermatic cord, the second leg extending around an opposite side of the spermatic cord and the first leg being affixed to the second leg;
(c) an elastic, compressible conical plug having a first end that is tapered extending at least partially into, and expanded to seat against at least a portion of a sidewall of, a deep inguinal ring and a second, opposing end of the plug that is substantially planar and mounted to the first face of the patch; and
(d) at least one fastener extending through the patch and into soft tissue near a pubic tubercle.

9. The hernia repair system in accordance with claim 8, wherein the mesh patch extends about 3 centimeters under the external oblique aponeurosis superiorly, about 3 centimeters lateral to the spermatic cord and deep inguinal ring, about 3 centimeters over the pubic tubercle region, and about 3 centimeters above the ilioinguinal ligament.
